(19) United States
(12) Patent Application Publication
(10) Pub. No.: US 2023/0053809 A1
(43) Pub. Date: Feb. 23, 2023

(54) SYSTEM AND METHOD FOR RECOGNITION OF HEALTH CONDITIONS ACCORDING TO IMAGE PROCESSING

(71) Applicant: Conflu3nce Ltd., Jerusalem (IL)

(72) Inventor: Tami Robyn Ellison, Jerusalem (IL)

(73) Assignee: Conflu3nce Ltd., Jerusalem (IL)

(21) Appl. No.: 17/890,289

(22) Filed: Aug. 18, 2022

Related U.S. Application Data
(60) Provisional application No. 63/235,099, filed on Aug. 19, 2021.

(51) Int. Cl. G06T 7/00; G16H 50/20

(52) U.S. Cl. CPC G06T 7/0012; G16H 50/20; G16H 40/67

(57) ABSTRACT

Disclosed herein is a method for identifying a health condition, comprising using at least one hardware processor for obtaining at least one image of tissue, identify edges of objects and of borders between colors in the at least one image, determine a threshold, divide images into a predetermined number of sections, juxtapose the predetermined number of sections in a non-contiguous manner, designate quadrants and rings on image, identify markers indicating detection of a health condition, generating a notification of identifying the health condition, and providing the notification to an output device.

### RELATED APPLICATIONS

The present application claims priority from U.S. provisional application Ser. No. 63/235,099, titled “SYSTEM AND METHOD FOR ANALYZING MEDICAL IMAGES” filed on Aug. 19, 2021, which in its entirety is incorporated herein by reference.

### FIELD OF THE INVENTION

The present disclosure generally relates to the field of health conditions based on image processing of lesions.

### BACKGROUND

Skin is the body's largest organ protecting internal organs from the external environment. Like the canary in the mine, skin can reflect internal health status through the manifestation of changes to the skin surface which are observable and trackable. For example, changes in the skin can serve as early indicators of Multiple Sclerosis, Parkinson's disease, and Alzheimer's disease and related Dementias.

Skin cancers are characterized by the abnormal growth of cells and are an ongoing medical concern for people around the world. One of the major issues is the impact of skin pigmentation on the accurate diagnosis of skin cancers, i.e., skin types of the patients. The incidence of the different types of skin cancer varies by skin pigmentation type. Melanoma, the most deadly form of skin cancer, tends to affect lighter skin types (Fitzsimmon Scale Type III or below) with Australia, New Zealand, European (Denmark, Netherlands, Switzerland), and Nordic (Sweden, Norway, Finland) populations consistently ranking at the top of the list. If detected early, melanoma and other skin cancers, such as Basal Cell Carcinoma (“BCC”) and Squamous Cell Carcinoma (“SCC”), along with Actinic Keratosis—considered to be a SCC precursor—are highly treatable.

FIG. 1 shows illustrated versions of various skin lesion attributes. Skin lesion characteristics are highly variable and are often pre-diagnostically categorized based on attributes. Illustration 150 shows typical lesions feature hyphenated into diagnostic classes. (Marghoob, A. A., Usatine, R. P., & Jaimes, N. (2013). Dermoscopy for the family physician. American family physician, 88 7, 441-50. Maui Derm, the Dermatology Meetings, 2019. https://mauiderm.com/2019-maui-derm-conference-resources/#tab-id-2).

Systems and methods for creating an image and/or automatically interpreting images are disclosed in U.S. Pat. Nos. 11,158,060, 11,176,675, which in their entirety are incorporated herein by reference.

Systems and methods for generating composite images are disclosed U.S. Pat. No. 10,582,189, which in its entirety is incorporated herein by reference.

A multi-purpose interactive cognitive platform is disclosed in U.S. Pat. Nos. 11,328,822, 11,298,062, which in their entirety are incorporated herein by reference.

### SUMMARY

The following embodiments and aspects thereof are described and illustrated in conjunction with systems, tools and methods which are meant to be exemplary and illustrative, not limiting in scope.

There is provided, in accordance with an embodiment, a method for identifying a health condition, including using one or more hardware processors for obtaining one or more images of tissue, identify edges of objects and of borders between colors in the one or more images, determine a threshold, divide images into a predetermined number of sections, juxtapose the predetermined number of sections in a non-contiguous manner, designate quadrants and rings on image, identify markers indicating detection of a health condition, generating a notification of identifying the health condition, and providing the notification to an output device.

In some embodiments, the processor is further configured to obtain a plurality of images, and reverse engineer the plurality of images to identify markers indicating the health condition at a previous point in time.

In some embodiments, the spatial landmarks and relationships are conserved between image parts, across the image, and between images for making comparisons over time.

In some embodiments, the processor is further configured to approximate edges thereby facilitating edge-to-edge symmetry, border regularity, color and diameter comparisons in the image.

In some embodiments, the designation of quadrants and rings introduces an artificial vertical edge—a discrete line of pixels at the interface that serves as a landmark for making edge-to-edge and quadrant comparisons of lesion parts.

In some embodiments, the processor is further configured to modify colors of the image to enhance the characteristics of the image.

In some embodiments, modifying colors reduces the color composition of the image.

There is further provided, in accordance with an embodiment, a system for identifying a health condition, including one or more servers configured to provide one or more images of a tissue, a client device including a user interface having one or more input devices and one or more output devices, and one or more processors configured to obtaining one or more images of tissue, identify edges of objects and of borders between colors in the one or more images, determine a threshold, divide the one or more images into a predetermined number of sections, juxtapose the predetermined number of sections in a non-contiguous manner, designate quadrants and rings on image, identify markers indicating detection of a health condition, generating a notification of identifying the health condition, and providing the notification to the one or more output device.

In some embodiments, the one or more processors are further configured to, obtain a plurality of images, reverse engineer the plurality of images to identify markers indicating the health condition at a previous point in time.

In some embodiments, spatial landmarks and relationships are conserved between image parts, across the image, and between images for making comparisons over time.

In some embodiments, the one or more processors are further configured to approximate edges in the image thereby facilitating edge-to-edge symmetry, border regularity, color and diameter comparisons in the image.

In some embodiments, the designation of quadrants and rings introduces an artificial vertical edge—a discrete line of pixels at the interface that serves as a landmark for making edge-to-edge and quadrant comparisons of lesion parts.

In some embodiments, the one or more processors are further configured to further configured to modify colors of the image to enhance the characteristics of the image.

In some embodiments, modifying colors reduces the color composition of the image.

There is further provided, in accordance with an embodiment, a computer program product for identifying a health condition, the computer program product including a non-transitory computer-readable storage medium having program code embodied therewith, the program code executable by one or more hardware processors to obtain at one or more images of tissue, identify edges of objects and of borders between colors in the one or more images, determine a threshold, divide images into a predetermined number of sections, juxtapose the predetermined number of sections in a non-contiguous manner, designate quadrants and rings on image, identify markers indicating detection of a health condition, generate a notification of identifying the health condition, provide the notification to an output device.

In some embodiments, the computer program product is further configured to obtain a plurality of images, reverse engineer the plurality of images to identify markers indicating the health condition at a previous point in time.

In some embodiments, the spatial landmarks and relationships are conserved between image parts, across the image, and between images for making comparisons over time.

In some embodiments, the computer program product is further configured to approximate edges thereby facilitating edge-to-edge symmetry, border regularity, color, and diameter comparisons in the image.

In some embodiments, the designation of quadrants and rings introduces an artificial vertical edge—a discrete line of pixels at the interface that serves as a landmark for making edge-to-edge and quadrant comparisons of lesion parts.

In some embodiments, the computer program product is further configured to modify colors of the image to enhance characteristics of the image.

In addition to the exemplary aspects and embodiments described above, further aspects and embodiments will become apparent by reference to the figures and by study of the following detailed description.

### BRIEF DESCRIPTION OF THE DRAWINGS

Some non-limiting exemplary embodiments or features of the disclosed subject matter are illustrated in the following drawings.

Identical, duplicate, equivalent, or similar structures, elements, or parts that appear in one or more drawings are generally labeled with the same reference numeral, optionally with an additional letter or letters to distinguish between similar entities or variants of entities, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspectives or from different points of view.

References to previously presented elements are implied without necessarily further citing the drawing or description in which they appear.

FIG. 1 shows a PRIOR ART schematic illustration of lesion attributes, according to certain exemplary embodiments;

FIG. 2 schematically illustrates a system configured to identify a health condition, according to certain exemplary embodiments;

FIG. 3 schematically illustrates a computerized device of the system in FIG. 2, according to certain exemplary embodiments;

FIG. 4 schematically illustrates a server of the system in FIG. 2, according to certain exemplary embodiments;

FIG. 5 outlines operations of a method for image processing for recognizing health conditions, according to certain exemplary embodiments;

FIG. 6 outlines operations of a method for image analysis of a set of images to reverse engineer lesion characteristics, according to certain exemplary embodiments;

FIGS. 7A-7C show color modification of a skin lesion image, according to certain embodiments;

FIGS. 8A-8J show image processing of skin lesion images, according to certain embodiments; and

FIGS. 9A-9B shows enhancement of a skin lesion, according to certain exemplary embodiments.

### DETAILED DESCRIPTION

A general non-limiting overview of practicing the present disclosure is presented below. The overview outlines the exemplary practice of embodiments of the present disclosure, providing a constructive basis for variant and/or alternative and/or divergent embodiments, some of which are subsequently described.

Described herein is a system and method for recognition of health conditions based on the processing of images of tissue, according to certain exemplary embodiments.

Skin pigmentation can serve as an important metric for detecting changes in skin health with pigmentation features associated with the interior of a lesion, around its edge, and relative to a background skin pigmentation. In developing strategies for analyzing skin features, traditional augmentation methods used for non-medical images can create authenticity issues, introducing hallucinatory and/or unrealistic representations. A subset of augmentation methods can be used which do not change pixel-level information such as rotation, flip, and image resizing. Similarly, pre-processing image filters methods used to highlight previously recognized or known Regions of Interest (“ROI”) can compromise input data by extracting seemingly “unnecessary” information; in this case enhancing lesion features but at the expense of background skin pigmentation information.

Furthermore, image datasets can have qualitative and quantitative issues, in part related to image sourcing and annotations. For example, an applied bias in annotations that follow traditional methods for evaluating lesions and elaborating through rule-in/rule-out differential algorithms applying Asymmetry, Border, Color, Diameter (“ABCD”) features within defined and variable thresholds but which are geared toward diagnosis and not early detection. While class-balancing can serve to equalize under-represented lesion categories, class balancing doesn't necessarily add true diversity to challenging atypical lesions, pigmented lesions on darker pigmented skin types, hypo and amelanotic lesions across all skin types, as well as historical images showing lesion progression which can help advance skin lesion analysis and our understanding of lesion progression.

From a health professional's perspective, lesion analysis combines both art and science—a “hands-on and minds-on” determination, even if initially made remotely through live telehealth consultations or store and forward interactions.

The utilization of artificial intelligence analysis can facilitate determining the health of certain tissues, such as skin health, as well as internal organs, such as the brain. Furthermore, applying computer vision methods can serve as an adjunct in supporting end-user consumers with early detection tools, and providers with analytical tools to track subtle changes over time thereby identifying lesion nuances and advancing predictive analytics in the field.

As will be described through certain exemplary embodiments, the system and method disclosed herein provide an agile image-based solution, by integrating image manipulation methods to: 1) support early detection of skin health changes by advancing logic-leaping pattern generalization and inference capabilities for image parts recognition; and, 2) develop an innovative generative adversarial model for identifying previously unrecognized predictive biomarkers of early/prodromal stage changes in skin health.

The system and method disclosed herein execute computer vision capabilities, which transform unseen data making unknown information known information thereby advancing predictive capabilities towards identifying skin health change biomarkers. Skin, the body's largest organ, while possessing the same fundamental structure is nonetheless unique in its external appearance and manifesting in health status changes over time, both within the same individual and across different populations. Qualitative and quantitatively useful dermatopathology datasets containing historical images of disease/condition progression are not always available, easily obtained, or may express highly individualized change pattern characteristics, including Asymmetry, Border, Color, Diameter, and Evolution/elevation (“ABCDE”) features in cancerous lesion patterns. Datasets can be biased, lacking an authentic diversity representative of real-world medical images able to support differential diagnosis or medical decision-making, contributing to a further exacerbation of health disparity issues. To address these challenges, the system and method disclosed herein present image processing operations for improving visual attention of image parts by manipulating and interrogating embedded image Gestalt characteristics, according to certain exemplary embodiments.

In some embodiments, the system and methods disclosed herein allow for the differentiation of lesion features which can be visualized using image enhancement filters with dermoscopy obtained polarized and non-polarized light images; evaluate lesions with sequential color-reductions that preserve interior lesion-edge-background pigmentation features; the identification of Lesion Factors (LF: (ABCDEF&G, primary/secondary morphologies, texture, location distribution, color, and other pattern characteristics) for developing lesion categories; visual correlate between LF and five (5 putative, characteristics-based lesion categories; and, knowledge transfer of “figure” attributes/edge contiguity characteristics in non-medical images.

Through the execution of Stitch and Peel operations which juxtapose non-adjacent image sections, logically pre-pooling and reducing image pixels across an image, computer visual attention can be focused on key image features, including edge characteristics to interrogate lesion ABCDE and pattern features with expert-level questions to improve recognition accuracy in affected tissue. Image manipulation tools will be used to cooperatively: 1) advance our understanding of parts recognition and parts-of-the-whole feature extraction by leveraging an image's Gestalt features; 2) develop skin lesion categories based on image characteristics, rather than diagnostic criteria or annotations; and, 3) design stitched chimeric constructs as source/target images in a GANs model for generating putative “known/unknown unknowns” biomarker change candidates.

In certain embodiments, these digital pathology tools are used to manipulate and evaluate image Gestalt characteristics, introducing top-down, supra-level changes designed to improve imaging inputs for feature extraction and image analysis. Ideally, by enriching the quality and quantity of lesion characteristics available for analysis, the methods will help deepen understanding of lesion progression and be able to focus attention on key Regions of Interest and hallmark changes/progressions in identifying early/earlier-stage skin health change biomarkers. In developing a new diagnostic-independent, features/characteristics-based classification system, the invention can re-define skin lesion characteristics and transform AI features' extraction and pattern analysis capabilities, giving consumer and provider stakeholders advanced visualization and early detection/early-warning tools to assess any skin lesion.

In some embodiments, an image of a lesion and/or image containing a “Region of Interest” of tissue is stitched and/or peeled, and the part and/or parts are assessed based on the image that was stitched and/or peeled. Stitching methods are configured to facilitate interior to edge comparisons, edge-to-edge analysis and figure element assessments relative to the ground elements, using embedded or constructed symmetry of the lesions, reducing pixel content while retaining spatial contexts. Lesions can be broadly defined as disruptions to the integrity of the tissue in which it is found and can constitute single cells, cell clusters, solid tumors and/or cancerous and/or benign growths regardless of location. In some embodiments, a figure position or ground position within an image of a lesion are determined, and the lesion is assessed based on the figure characteristics and/or ground characteristics determined and or their relative positions. Alternatively or additionally, the images of the lesion may be analyzed for color blocks and/or edges and/or horizon-type contiguities, analyzing and/or comparing interior, edge and background (ground) features which can be used to assess a lesion.

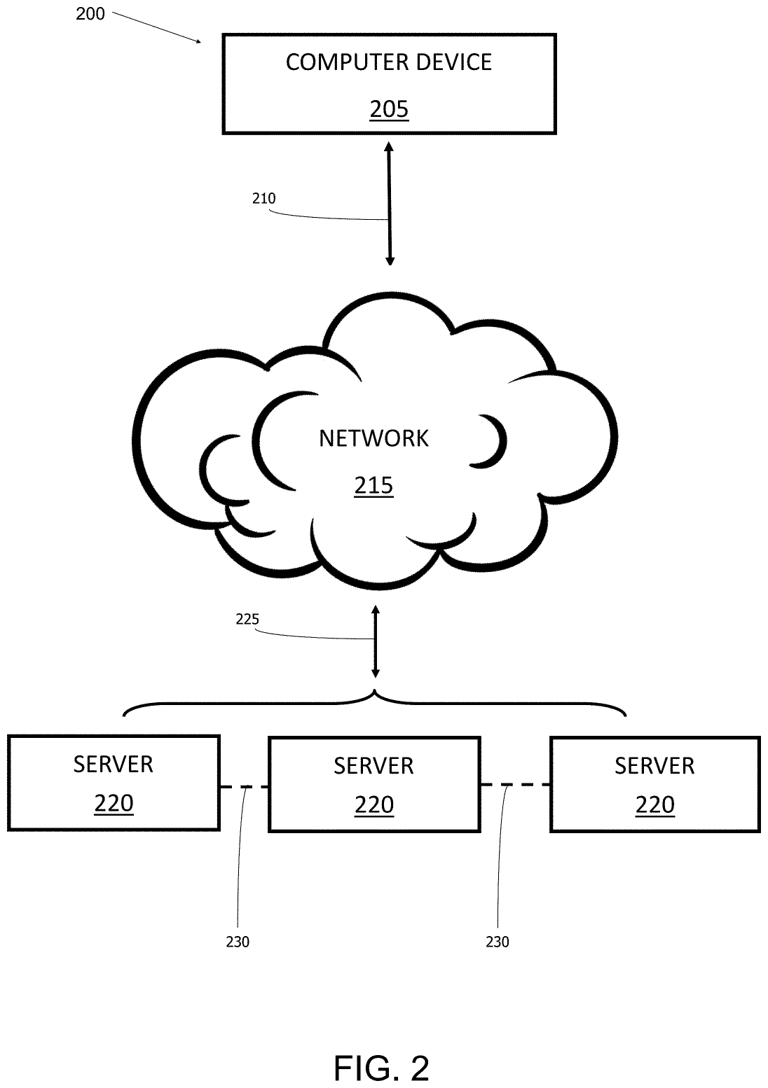

FIG. 2 schematically illustrates a system 200 configured for recognizing abnormalities in tissue and providing an indication of a health condition, according to certain exemplary embodiments. For example, system 200 may analyze images, and apply image manipulation modules to enhance and expedite recognition of abnormal tissue. The image analysis can include leveraging embedded multiple Gestalt principles, (figure-ground, closure, continuation), engaging top-down cognition and bottom-up sensory processing, to virtually reassemble portions of one or more images through virtual reconstruction of the intact image and of the image portion occupying the ground position.

System 200 can include a computerized device 205 communicating with one or more servers 220 illustrated as three instances of servers 220, representing any number of servers 220, as indicated by dashed line 230. In some embodiments, computerized device 205 can be a smartphone, a laptop, a desktop, a tablet, or the like. Computerized device 205 is connected to a network 215 by any communication facility or facilities included in system 200 as schematically illustrated by arrow 210. Servers 220 are connected to network 215 by any communication facility or facilities included in system 200 as illustrated by arrow 225. Communication facilities 210, 225 facilitate communication between computerized device 205 and servers 220.

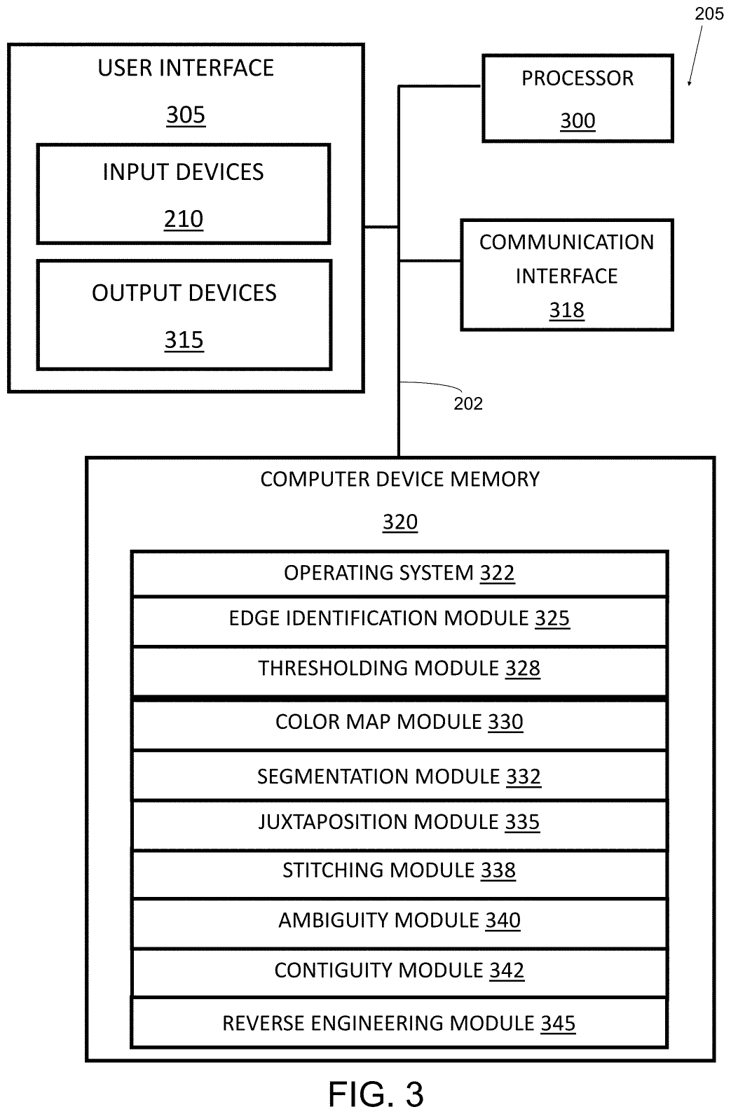

FIG. 3 schematically illustrates computerized device 205, according to certain exemplary embodiments. Computerized device 205 includes one or more processors 300, one or more communication interfaces 318, computer device memory 320 and one or more communication buses 302 for interconnecting these components. One or more communication interfaces 318 can include a wired and/or wireless interface for receiving data from and/or transmitting data to one or more servers 220 (FIG. 2).

In some implementations, data communications are carried out using any of a variety of custom or standard wireless protocols (e.g., NFC, RFID, IEEE 802.15.4, Wi-Fi, ZigBee, 6LoWPAN, Thread, Z-Wave, Bluetooth, ISA100.11a, WirelessHART, MiWi, etc.). Furthermore, in some implementations, data communications are carried out using any of a variety of custom or standard wired protocols (e.g., USB, Firewire, Ethernet, etc.). For example, the one or more communication interfaces 318 include a wireless interface for enabling wireless data communications with servers 220 and/or other wireless (e.g., Bluetooth-compatible) devices. Furthermore, in some implementations, the wireless interface (or a different communications interface of the one or more communication interfaces 318) enables data communications with other WLAN-compatible devices.

Computer device memory 320 includes high-speed random-access memory, such as DRAM, SRAM, DDR RAM, or other random-access solid-state memory devices; and may include non-volatile memory, such as one or more magnetic disk storage devices, optical disk storage devices, flash memory devices, or other non-volatile solid-state storage devices. Computer device memory 320 may optionally include one or more storage devices remotely located from the processor 300. Computer device memory 320, or alternately, the non-volatile memory within computer device memory 320, includes a non-transitory computer-readable storage medium. In some implementations, computer device memory 320 or the non-transitory computer-readable storage medium of computer device memory 320 stores the following programs, modules, and data structures, or a subset or superset thereof:

an operating system 322 that includes procedures for handling various basic system services and for performing hardware-dependent tasks;

an edge identification module 325 to identify an edge or border region surrounding a portion of tissue of interest that is to be isolated and processed. For example, identifying the edges of a skin abnormality in an image.

a threshold module 328 for determining a threshold of color difference between the tissue and the surrounding skin. In some embodiments, threshold module 335 is executed concurrently edge identification module 325

a color map module 330 to modify colors of the image or to identify different color pixels in the image.

a segmentation module 332 to segment and divide the image according to pixel density differences in the images. Segmentation module 332 is configured to match a segmented image with an unsegmented image thereby isolating the tissue portion of interest. Segmentation module 332 is configured to split the isolated image into a predetermined number of sections, such as three sections.

a juxtaposition module 335 to juxtapose the predetermined number of sections thereby reducing the size of the image that has to be analyzed by processor 305.

an ambiguity module 338 to enlarge and enhance an image for representation purposes and to facilitate determining the characteristics of the tissue. In some embodiments ambiguity module 338 may execute a Sobel edge analysis and invert the colors to facilitate detecting internal asymmetries in the predetermined segments.

a stitch and peel module 340 to split the isolated image into quadrants and apply equally spaced, concentric rings to the isolated image thereby allowing for tissue edge comparisons of borders, color, and other relevant characteristics. In some embodiments, multiple comparative images can be made between concentric rings and quadrants for color and content identity and differences. In some embodiments, sub-quadrants, two-dimensional and three-dimensional, measurements can be recorded to detect subtle changes in the tissue.

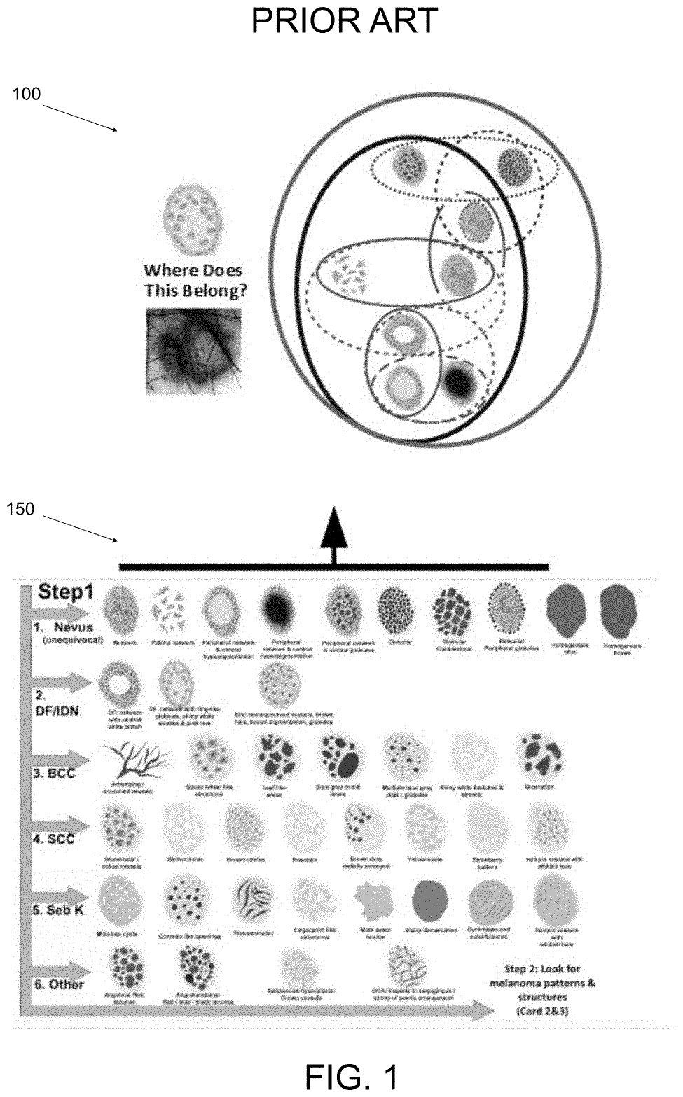

a reverse engineering module 350 to facilitate identifying tissue features at earlier points in time that indicate a health condition to ensure faster treatment. Reverse engineering module 350 enables obtaining lesion characteristics for identifying earlier-stage biomarkers based on GANs chimera-generated candidates model and identification of differentiated features and which can be matched to the detection of early-stage biomarkers, when available. In developing robust image categories based on lesion features with ranged value correlates, the illustrations-developed categories, as illustrated in FIG. 1, can be matched to “wild” lesions and be used to identify outliers and previously unseen/unidentified image category patterns. In some embodiments, reverse engineering module 350 can obtain images of other tissues having the characteristics of the health condition from which processor 300 can generate an algorithm for identifying a health condition of a tissue, for example, a lesion on skin. Thereby, reverse engineering module 350 can roll back the early detection timeline at which images may be available of the tissue to facilitate identifying unknown biomarkers of disease and improving health outcomes with new research and design discoveries.

In some embodiments, where historical and/or progression images of tissue are unavailable whether from the same patients, multiple patients, in a dataset, and/or to build potential early-stage and/or progression characteristics, a generative model such as StyleGans may be used. Chimeric and/or half-constructs can be used as source and/or targets for generative modeling, and/or using a features-driven tool for effecting targeted changes to individual attributes. In some embodiments, chimeric stitched constructs would combine different percentages of Normal (N) and Abnormal (Abn) image parts (source image), from the same or different categories/clusters and with the reversed percentages in target images (0.7N:0.3Abn→0.3N:0.7Abn). In some embodiments, the chimera construct can include a gap, an uneven percentage size between the source and the target (Source 0.6N:---0.3Abn→Target 0.3N:0.7Abn) to generate additional output images with different transition image attributes.

In reverse engineering tissue maladies to their point of origin, progenitor cells might be classed into two general categories: 1) those emerging from an existing skin feature such as a mole, scar, nevus, irregular patch, and pigmented area; and, 2) lesions appearing in areas without any noticeable and/or visual manifestation. The underlying dermis and the interplay between other skin structures, specifically melanosomes which show different expression and distribution patterns based on skin pigmentation types among other factors that can provide insights into skin integrity issues, cancer development, and systems failure and product defects using new detection tools and devices as knowledge continues to evolve.

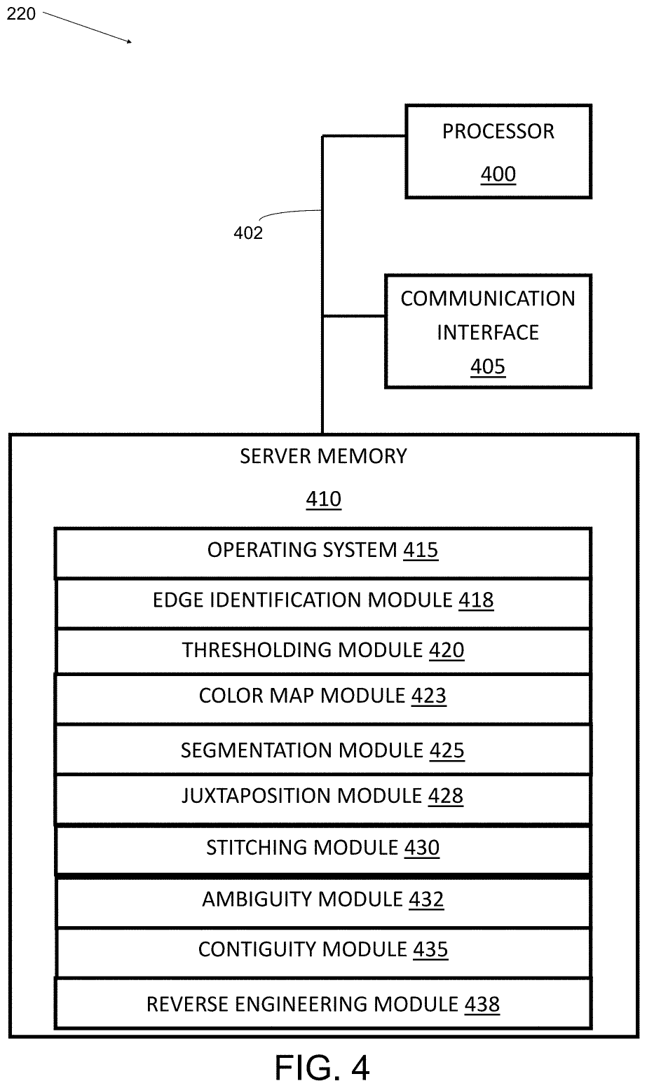

FIG. 4 schematically illustrates server 220, according to certain exemplary embodiments. Server 220 includes one or more processors 400, one or more communication interfaces 405, server memory 410 and one or more communication buses 402 for interconnecting these components. One or more communication interfaces 405 can include a wired and/or wireless interface for receiving data from and/or transmitting data to computerized device 205.

In some implementations, data communications are carried out using any of a variety of custom or standard wireless protocols (e.g., NFC, RFID, IEEE 802.15.4, Wi-Fi, ZigBee, 6LoWPAN, Thread, Z-Wave, Bluetooth, ISA100.11a, WirelessHART, MiWi, etc.). Furthermore, in some implementations, data communications are carried out using any of a variety of custom or standard wired protocols (e.g., USB, Firewire, Ethernet, etc.). For example, the one or more communication interfaces 405 include a wireless interface for enabling wireless data communications with computerized device 205 and/or or other wireless (e.g., Bluetooth-compatible) devices. Furthermore, in some implementations, the wireless interface (or a different communications interface of the one or more communication interfaces 405) enables data communications with other WLAN-compatible devices.

Server memory 410 includes high-speed random-access memory, such as DRAM, SRAM, DDR RAM, or other random-access solid-state memory devices; and may include non-volatile memory, such as one or more magnetic disk storage devices, optical disk storage devices, flash memory devices, or other non-volatile solid-state storage devices. Server memory 410 may optionally include one or more storage devices remotely located from the processor 400. Server memory 410, or alternately, the non-volatile memory within server memory 410, includes a non-transitory computer-readable storage medium. In some implementations, server memory 410 or the non-transitory computer-readable storage medium of server memory 410 stores the following programs, modules, and data structures, or a subset or superset thereof:

an operating system 415 that includes procedures for handling various basic system services and for performing hardware-dependent tasks.

an edge identification module 418 to identify an edge or border region surrounding a portion of tissue of interest that is to be isolated and processed. For example, identifying the edges of a skin abnormality in an image.

a threshold module 420 for determining a threshold of color difference between the tissue and the surrounding skin. In some embodiments, threshold module 420 is executed concurrently edge identification module 418

a color map module 423 to modify colors of the image or to identify different color pixels in the image.

a segmentation module 425 to segment the image based on a pixel density difference in the images. Segmentation module 425 is configured to match a segmented image with an unsegmented image thereby isolating the tissue portion. Segmentation module 425 is configured to split the isolated image into a predetermined number of sections, such as three sections.

a juxtaposition module 428 to juxtapose the predetermined number of sections thereby reducing the size of the image that has to be analyzed by processor 305.

an ambiguity module 430 to enlarge and enhance an image for representation purposes and to facilitate determining the characteristics of the tissue. In some embodiments ambiguity module 430 may execute a Sobel edge analysis and invert the colors to facilitate detecting internal asymmetries in the predetermined segments.

a stitch and peel module 435 to designate quadrants and equally spaced, concentric rings to the isolated image thereby allowing for tissue edge comparisons of borders, color, and other relevant characteristics. In some embodiments, multiple comparative images can be made between concentric rings and quadrants for color and content identity and differences. In some embodiments, sub-quadrants, two-dimensional and three-dimensional, measurements to detect subtle changes in the tissue.

a reverse engineering module 438 to facilitate identifying tissue features at earlier points in time that indicate a health condition to ensure faster treatment. Reverse engineering module 438 enables obtaining lesion characteristics for identifying earlier-stage biomarkers based on GANs chimera-generated candidates model and identification of differentiated features and which can be matched to the detection of early-stage biomarkers, when available. In developing robust image categories based on lesion features with ranged value correlates, the illustrations-developed categories can be matched to “wild” lesions and be used to identify outliers and unseen image category patterns. In some embodiments, reverse engineering module 438 can obtain images of other tissues having the characteristics of the health condition from which processor 400 can generate an algorithm for identifying a health condition of a tissue, for example, a skin lesion. Thereby, reverse engineering module 438 can roll back the early detection timeline at which images of the tissue may be available to facilitate identifying unknown biomarkers of disease and improving health outcomes with new research and design discoveries.

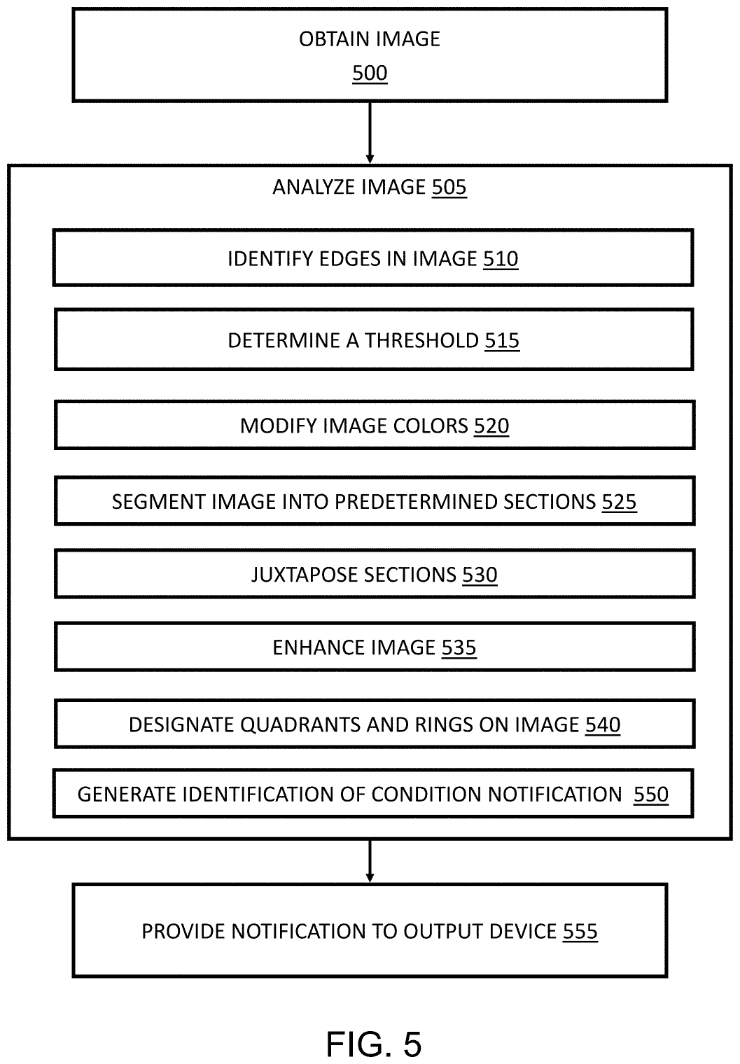

FIG. 5 outlines operations of a method for image analysis to recognize a lesion, according to certain exemplary embodiments. In operation 500, processor 300 or 400 obtains an image.

In operation 505, processor 300 or 400 analyzes the image to identify a condition in a tissue, for example, identifying a skin lesion. Operation 505 includes multiples operations as follows:

In operation 510, processor 300 or 400 identifies edges of an object in an image. In some embodiments, Gestalt image analysis of tissue facilitates identifying edges and borders of objects in the image. Processor 300 or 400 identifies image characteristics and an understanding of the multiplicity of interactions and the hierarchical relationship of image parts within images and between images. Recognizing and understanding the role and contribution of an image's contiguity features—edges, horizons, and color blocks—to figure (foreground) and ground (background) dynamics and depth perception is applicable to both human cognition, computer vision, and two-dimensional (“2D”) to three-dimensional (“3D”) environment translation.

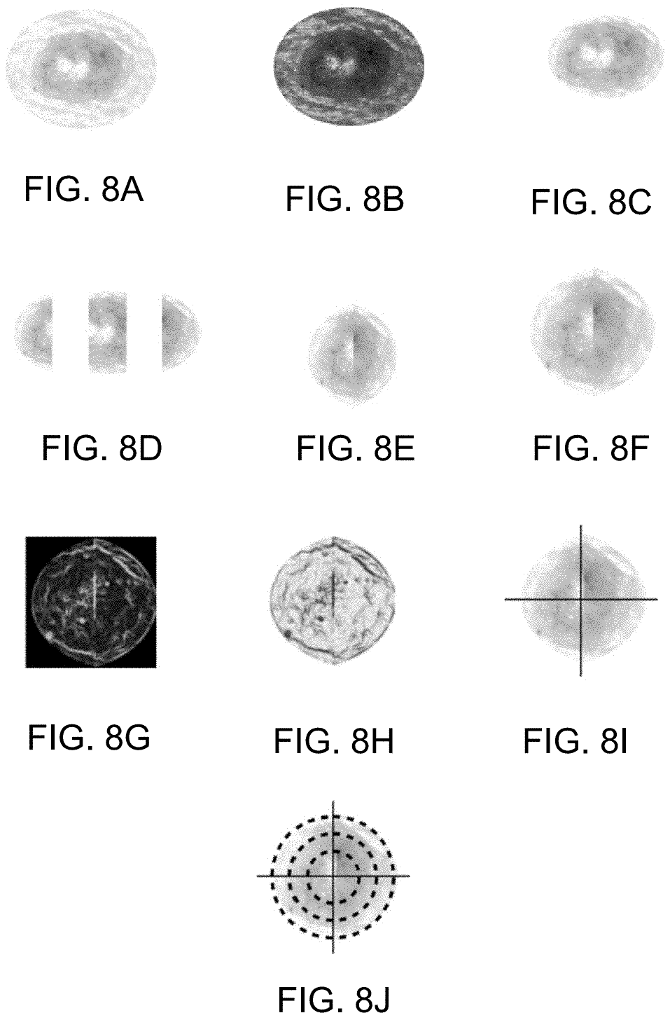

In operation 515, processor 300 or 400 determine a threshold for an image as shown in FIG. 8A. Image datasets can have qualitative and quantitative issues, in part related to image sourcing and annotations—an applied bias that follows traditional methods for evaluating lesions and elaborating through rule-in/rule-out differential algorithms applying ABCD features within defined and variable thresholds but which are geared toward diagnosis and not early detection. While class-balancing can serve to equalize under-represented lesion categories, class balancing doesn't necessarily add true diversity to challenging atypical lesions, pigmented lesions on darker pigmented skin types, hypo and amelanotic lesions across all skin types, as well as historical images showing lesion progression which can help advance skin lesion analysis and identifying lesion progression.

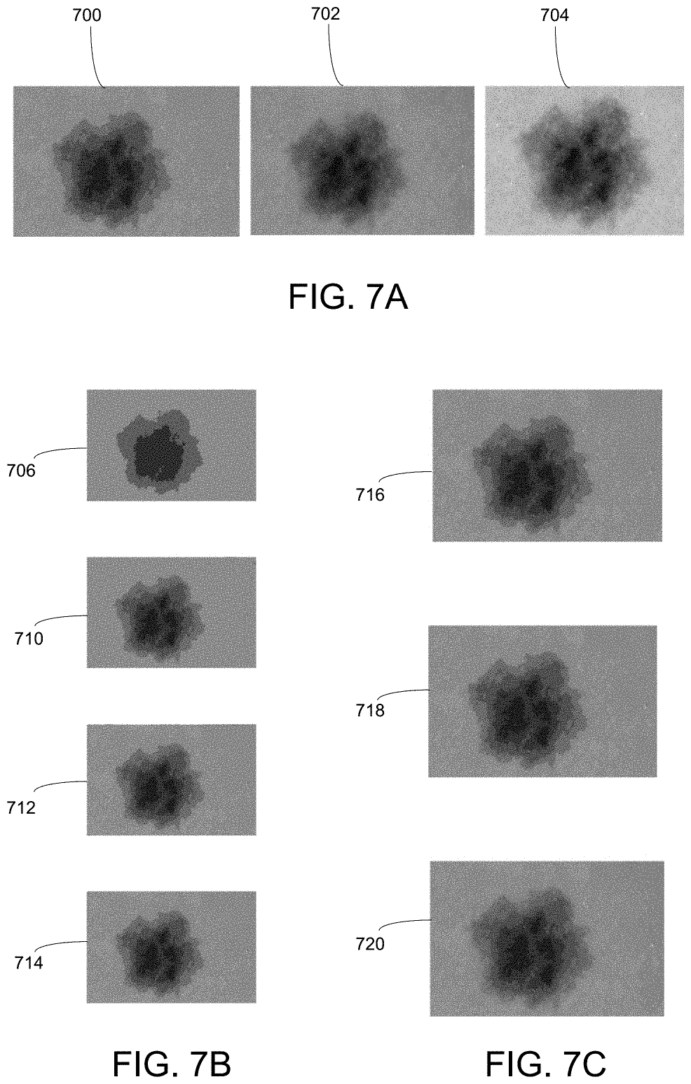

In operation 520, processor 300 or 400 modify the colors in the image thereby emphasizing borders and different regions in the image as shown in FIGS. 7A-7C. In some embodiments, the image obtained in step 500 is of low quality or has distortions, the colors of the image can be modified by making the image darker or brighter to enhance regions of the image.

In operation 525, processor 300 or 400 segments the image into predetermined sections, such as into three sections as shown in FIG. 8D.

In some embodiments, as shown in FIGS. 7B-7C lesion measurements can include an image segmentation step with a reduction in color composition to 4, 6, 8, 10, or more colors but less than the original number of colors represented in the lesion, depending on the complexity of the lesion, and which can be followed by treatment of the image with or without additional edge filters such as Sobel. In one embodiment color reduction can be used to compare lesion features while conserving background pigmentation characteristics in the area surrounding the lesion. In one embodiment, the color-reduced images can be used to evaluate lesion size, edge characteristics, and lesion coherency measured against norms and developed ranges for progressed lesions, wounds, or other presentations. In one embodiment, stitching can be applied to the image to facilitate size, edge, color, and coherency assessments where stitching can be left to right; right to left, top to bottom, bottom to top, or performed across one or more diagonals to juxtapose different combinations of image parts for point-in-time and change comparisons. Comparisons over time and/or between lesions can be presented as a series of color-reduced overlays, aligning point-in-time images, or tracking changes over time.

In operation 530, processor 300 or 400 juxtaposes the segments as shown in FIG. 8E. The segments are juxtaposed such that non-contiguous regions of the image are approximated thereby reducing the size of the image and the details of the image that have to be analyzed by processor 300 or 400 and to logically pre-pool pixel content, facilitating pan-lesion characteristics analysis. This facilitates creating an internal crop that logically punctuates an image, juxtaposing non-contiguous regions to interrupt an image's “native” content fluency and structural confluency. The stitch introduces an artificial vertical edge—a discrete line of pixels at the interface that serves as a landmark for making edge-to-edge and quadrant comparisons across the stitch and refining ABCDE lesion analysis. Spatial landmarks and relationships are conserved between image parts, across the image, and between images for making comparisons over time. Stitched image constructs can be generated with a single step or multiple stitches and peel steps, and can be vertical (left to right) or horizontal (top to bottom), approximating edges and facilitating edge-to-edge symmetry, regularity, color, diameter (ABCD features) comparisons and tracking of changes over time. In some embodiments, ideal Sn—P constructs may efficiently resolve contiguity disruptions to a PixelParts(min)—an endpoint requiring minimal touchpoint preprocessing; not requiring directed training with new image sets; and, enhancing computer vision-related tasks without compromising spatial contexts. Further, multiple Sn—P constructs could cooperatively be used across image scenes with smaller and/or larger peels for developing greater overall scene understanding effectively doing more with less.

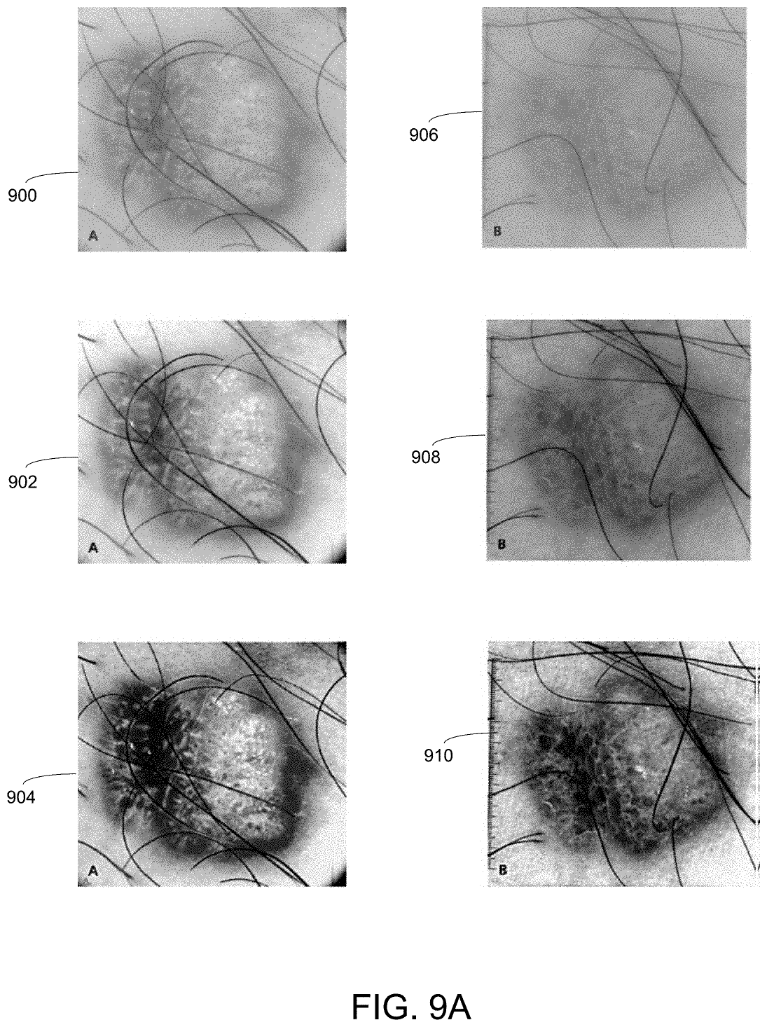

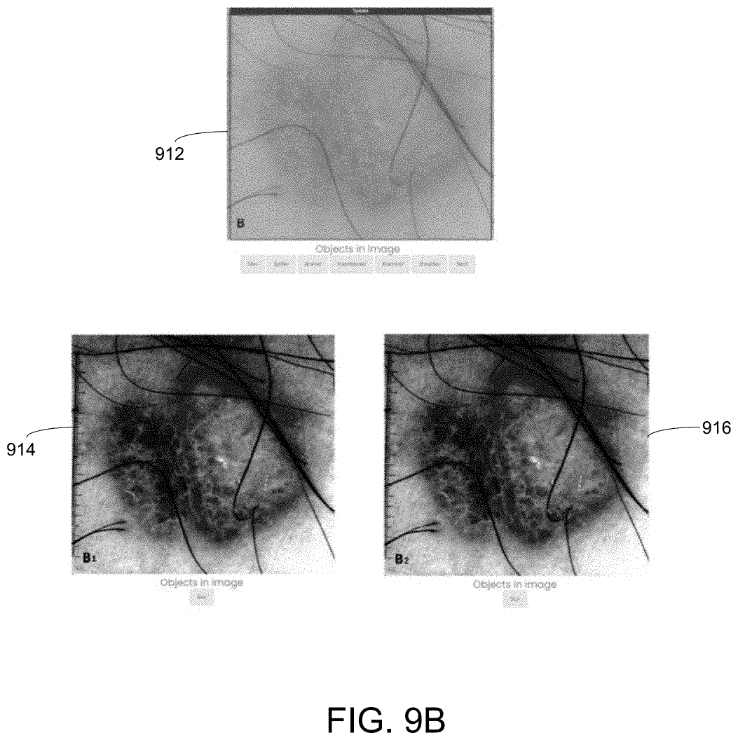

In operation 535, processor 300 or 400 enhance the image to facilitate identification, visualization, and analytical capabilities with detailed images as shown in FIGS. 9A-9B. In some embodiments, a non-polarized image 906 is enhanced with polarizing light 900. In some embodiments the images are processed with a white balance filter as shown in images 902, 904. In some embodiments, the image is processed using a Reitnex filter as shown in images 904 and 908. In some embodiments, an original image 912 is processed using a Retinex filter to achieve a halftone image 914 and a full-color image 916 which are then processed using a sandboxed computer vision model. For example, when original image 912 is analyzed by processor 300 or 400, it may be considered to show a spider rather than a spot on the skin, whereas the enhancement shown in images 914, 916 processor 300 or 400 can identify the spot as being part of skin.

In operation 540, processor 300 or 400 designate quadrants and rings as shown in FIGS. 81-8J.

In operation 550, processor 300 or 400 generates a notification of whether a condition has been detected in the tissue according to the image. In some embodiments, the notification is the image with markings showing the identified portions that may be problematic. In some embodiments, the notification can be a message stating that a health condition is present.

In operation 555, processor 300 or 400 provides notification to output devices 315 (FIG. 3).

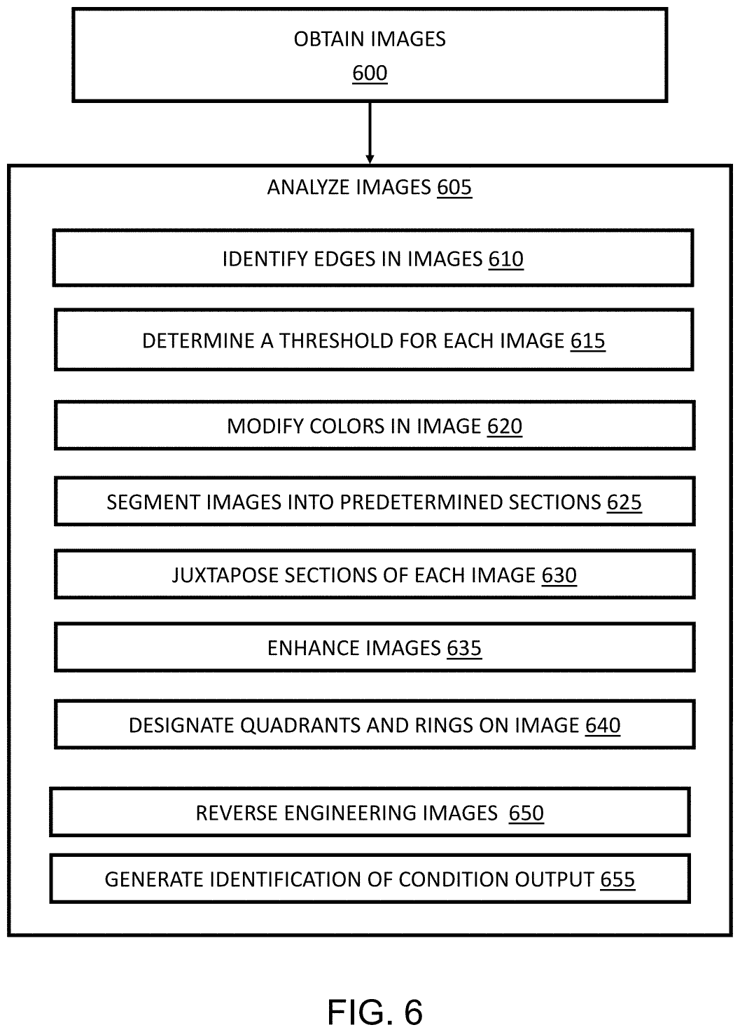

FIG. 6 outlines operations of a method for image analysis of a set of images to reverse engineer lesion characteristics, according to certain exemplary embodiments. In operation 600, processor 300 or 400 obtains one or more images. The images can include images of a tissue, such as a skin lesion over a predetermined window of time. In operation 605, processor 300 or 400 analyzes the image to identify a condition in a tissue, for example, identifying a lesion on skin. Operation 605 includes operations 610, 615, 620, 625, 630, 635, 640, 645 and 655 are performed, on the images, which correspond to operations 510, 515, 520, 525, 530, 535, 540, 550 of FIG. 5. Operation 605 furthermore includes operation 650, in which processor 300 or 400 reverse engineers the images to detect whether an indication presented itself at a previous time or an earlier timepoint when an image was taken.

In the context of some embodiments of the present disclosure, by way of example and without limiting, terms such as ‘operating’ or ‘executing’ imply also capabilities, such as ‘operable’ or ‘executable’, respectively.

Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor or a micro-processor, RISC processor, or DSP, possibly including additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry.

The processor and program may constitute the same apparatus, at least partially, such as an array of electronic gates, such as FPGA or ASIC, designed to perform a programmed sequence of operations, optionally including or linked with a processor or other circuitry.

The term computerized apparatus or a computerized system or a similar term denotes an apparatus including one or more processors operable or operating according to one or more programs. As used herein, without limiting, a module represents a part of a system, such as a part of a program operating or interacting with one or more other parts on the same unit or on a different unit, or an electronic component or assembly for interacting with one or more other components.

As used herein, without limiting, a process represents a collection of operations for achieving a certain objective or an outcome.

As used herein, the term ‘server’ denotes a computerized apparatus providing data and/or operational service or services to one or more other apparatuses.

The term ‘configuring’ and/or ‘adapting’ for an objective, or a variation thereof, implies using at least a software and/or electronic circuit and/or auxiliary apparatus designed and/or implemented and/or operable or operative to achieve the objective.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, illustrated or described operations may occur in a different order or in combination or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ or ‘close’ implies at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values.

As used herein, ellipsis ( . . . ) between two entities or values denotes an inclusive range of entities or values, respectively. For example, A . . . Z implies all the letters from A to Z, inclusively.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded.

Terms in the claims that follow should be interpreted, without limiting, as characterized or described in the specification.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method for identifying a health condition, comprising using at least one hardware processor for:
obtaining at least one image of tissue;
identifying edges of objects and of borders between colors in said at least one image;
determining a threshold;
dividing images into a predetermined number of sections;
juxtaposing said predetermined number of sections in a non-contiguous manner;
designating quadrants and rings on image;
identifying markers indicating detection of a health condition;
generating a notification of identifying the health condition;
providing the notification to an output device.

2. The method according to claim 1, further comprising using at least one hardware processor for:
obtaining a plurality of images;
reversing engineer the plurality of images to identify markers indicating the health condition at a previous point in time.

3. The method according to claim 2, wherein spatial landmarks and relationships are conserved between image parts, across the image, and between images for making comparisons over time.

4. The method according to claim 1, further comprising using at least one hardware processor for approximating edges thereby facilitating edge-to-edge symmetry, border regularity, color and diameter comparisons in the image.

5. The method according to claim 1, wherein said designation of quadrants and rings introduces an artificial vertical edge—a discrete line of pixels at the interface that serves as a landmark for making edge-to-edge and quadrant comparisons of lesion parts.

6. The method according to claim 1, further comprising using at least one hardware processor for modifying colors of the image to enhance the characteristics of the image.

7. The method according to claim 6, wherein said modifying colors reduces the color composition of the image.

8. A system for identifying a health condition, comprising:
at least one server configured to provide at least one image of a tissue;
a client device comprising:
a user interface having at least one input device and at least one output device; and,
at least one processor configured to:
obtain at least one image of tissue;
identify edges of objects and of borders between colors in said at least one image;
determine a threshold;
divide images into a predetermined number of sections;
juxtapose said predetermined number of sections in a non-contiguous manner;
designate quadrants and rings on image;
identify markers indicating detection of a health condition;
generate a notification of identifying the health condition; and,
provide the notification to said at least one output device.

9. The method according to claim 8, wherein said at least one processor is further configured to:
obtain a plurality of images;
reverse engineer the plurality of images to identify markers indicating the health condition at a previous point in time.

10. The method according to claim 9, wherein spatial landmarks and relationships are conserved between image parts, across the image, and between images for making comparisons over time.

11. The method according to claim 8, wherein said at least one processor is further configured to approximate edges in the image thereby facilitating edge-to-edge symmetry, border regularity, color and diameter comparisons in the image.

12. The method according to claim 10, wherein said designation of quadrants and rings introduces an artificial vertical edge—a discrete line of pixels at the interface that serves as a landmark for making edge-to-edge and quadrant comparisons of lesion parts.

13. The method according to claim 10, wherein said at least one processor is further configured to modify colors of the image to enhance the characteristics of the image.

14. The method according to claim 13, wherein said modifying colors reduces the color composition of the image.

15. A computer program product for identifying a health condition, the computer program product comprising a non-transitory computer-readable storage medium having program code embodied therewith, the program code executable by at least one hardware processor to:
obtain at least one image of tissue;
identify edges of objects and of borders between colors in said at least one image;
determine a threshold;
divide images into a predetermined number of sections;
juxtapose said predetermined number of sections in a non-contiguous manner;
designate quadrants and rings on image;
identify markers indicating detection of a health condition;
generate a notification of identifying the health condition;
provide the notification to an output device.

16. The computer program product according to claim 15, further configured to:
obtain a plurality of images;
reverse engineer the plurality of images to identify markers indicating the health condition at a previous point in time.

17. The computer program product according to claim 16, wherein spatial landmarks and relationships are conserved between image parts, across the image, and between images for making comparisons over time.

18. The computer program product according to claim 15, further configured to approximate edges thereby facilitating edge-to-edge symmetry, border regularity, color, and diameter comparisons in the image.

19. The computer program product according to claim 15, wherein said designation of quadrants and rings introduces an artificial vertical edge—a discrete line of pixels at the interface that serves as a landmark for making edge-to-edge and quadrant comparisons of lesion parts.

20. The computer program product to claim 15, further configured to modify colors of the image to enhance characteristics of the image.
